(19) United States
(12) Patent Application Publication
(10) Pub. No.: US 2020/0188601 A1
(43) Pub. Date: Jun. 18, 2020

(54) MAGNETIC DOSAGE SENSING

(71) Applicant: Verily Life Sciences LLC, South San Francisco, CA (US)

(72) Inventors: Louis H. Jung, Foster City, CA (US); William Biederman, San Francisco, CA (US)

(21) Appl. No.: 16/218,324

(22) Filed: Dec. 12, 2018

(51) Int. Cl. A61M 5/315

(52) U.S. Cl. CPC A61M 5/31568; A61M 5/31525; A61M 2005/3126; A61M 2205/3317; A61M 2205/52; A61M 5/31533

(57) ABSTRACT

An attachment for a drug injection pen includes a housing shaped to attach to a proximal end of the drug injection pen, and a dosage measurement system disposed at least in part within the housing. The dosage measurement system is coupled to receive a rotational motion, from a dosage injection mechanism disposed within the drug injection pen, when the drug injection pen dispenses a fluid. The dosage measurement system includes one or more magnets to generate a magnetic field, and one or more magnetic field sensors. The one or more magnetic field sensors rotate relative to the one or more magnets when the drug injection pen dispenses a fluid, and output a signal indicative of the magnetic field.

### TECHNICAL FIELD

This disclosure relates generally to drug injection and in particular but not exclusively, relates to tracking injection quantities.

### BACKGROUND INFORMATION

Measuring the quantity and recording the timing of a drug's administration is an integral part of many disease treatments. For many treatments, to achieve the best therapeutic effect, specific quantities of a drug may need to be injected at specific times of day. For example, individuals suffering from diabetes may be required to inject themselves regularly throughout the day in response to measurements of their blood glucose. The frequency and volume of insulin injections must be carefully tracked and controlled to keep the patient's blood glucose level within a healthy range.

Currently, there are a limited number of methods or devices capable of tracking drug administration without requiring the user to manually measure and record the volume, date, and time. A variety of glucose injection syringes/pens have been developed, but there is much room for significant advancement in the technology in order to reduce the size, lower the cost, enhance the functionality, and improve the accuracy. Thus, the current technology may not be an ideal long-term solution. For example, current insulin pens are often disposable, but do not include dosage tracking. A smaller portion of the market is composed of reusable pens which are more expensive, and still do not include accurate dosage-tracking capabilities.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. The drawings are not necessarily to scale, emphasis instead being placed upon illustrating the principles being described.

FIG. 1 illustrates an injection pen system, in accordance with an embodiment of the disclosure.

FIGS. 2A-2B illustrate an exploded view of the pen button depicted in FIG. 1, in accordance with an embodiment of the disclosure.

FIGS. 3A-3D illustrate several examples of magnet and magnetic sensor configurations that may be used within the pen button of FIG. 2, in accordance with several embodiments of the disclosure.

FIG. 4 illustrates an example tunnel magnetoresistance (TMR) device architecture, in accordance with an embodiment of the disclosure.

FIG. 5 is a flow chart illustrating a method of dispensing of fluid from a drug cartridge, in accordance with several embodiments of the disclosure.

### DETAILED DESCRIPTION

Embodiments of an apparatus, system, and method for magnetic dosage sensing are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

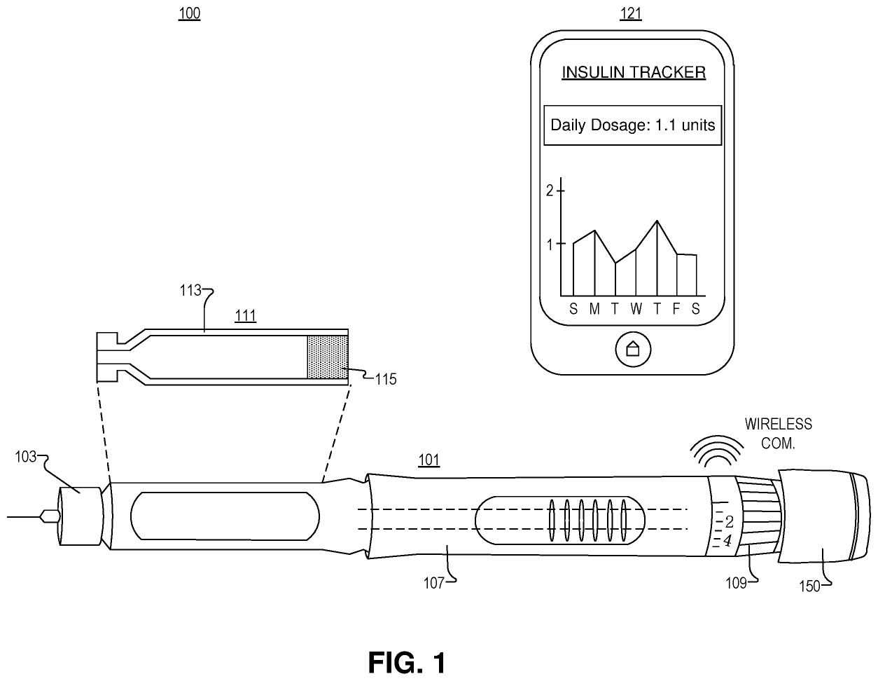

In an injection pen (like the one depicted in FIG. 1), there may be many moving components (e.g., a system to dial a dosage, a lead screw to expel the dosage from the medication cartridge, etc.). A magnetic angular sensor may be used to determine the absolute (rotational) position of some of the rotating components within the pen. As will be explained in greater detail below, knowing the position of these moving parts within the injection pen may be used to track a dosage (volume) of fluid dispensed from the pen.

In some embodiments, the pen may include a printed circuit board (PCB) that rotates. The magnetic sensor may be used to determine the absolute rotational position of the PCB relative to a diametric disc magnet or two axial magnets. In some embodiments, the magnetic angular sensor may be a tunnel magnetoresistance (TMR) device.

A TMR element is a thin-film element having a barrier layer made of a thin insulator sandwiched between two ferromagnetic layers (free layer and a fixed layer). Although the magnetization direction of the fixed layer is fixed, the magnetization of the free layer changes with the rotation of the magnet (which is, in turn, rotating with the button during an ejection).

The electrical resistance of the TMR element changes along with the change in the magnetization direction in the free layer. The electrical resistance is smallest when the magnetization directions of the fixed layer and free layer are parallel, causing a large current to flow into the barrier layer. When the magnetization directions are antiparallel, the resistance becomes extremely large, and little current flows into the barrier layer. Accordingly, the change in resistance (or change in voltage across the TMR element) can be measured as a sinusoidal wave over time. In some embodiments, this device can be made using a custom integrated circuit that will be mounted onto the bottom of the PCB.

As will be shown, a two-dimensional TMR based angular sensor and custom analog front-end (AFE) controller may be used to sense absolute angular orientation of an on-axis 2-pole magnet rotating over the center of the board. Similarly, a two-dimensional TMR sensor may include two one-dimensional sensors, which are placed at 90 degrees to each other. Each 1D sensor outputs a sinusoidal wave. These waves are 90 degrees out of phase, i.e., quadrature encoded. It is appreciated that either the board or the magnet can be rotating relative to the grip (e.g., the portion of the pen that the user holds). When the person presses the pen button to dispense the fluid, the measurement process (e.g., the controller/processor detecting that the button is pressed) may start. The battery in the pen drives the current across the TMR.

The following disclosure will discuss the embodiments described above, and other embodiments, as they relate to the figures.

FIG. 1 illustrates an injection pen system 100, in accordance with an embodiment of the disclosure. Pen system 100 includes injection pen 101, drug cartridge 111, and processing device 121 (e.g., a smart phone).

Drug cartridge 111 includes cartridge body 113, and plunger head 115. In the depicted embodiment, plunger head 115 starts near the rear of drug cartridge 111 and is pushed forward in drug cartridge 111 (with a dosage injection mechanism—shown as dashed lines—disposed in injection pen 101). This forces medication/fluid out of the narrow end of drug cartridge 111 when a user chooses to dispense a fluid. In one embodiment, cartridge body 113 includes borosilicate glass.

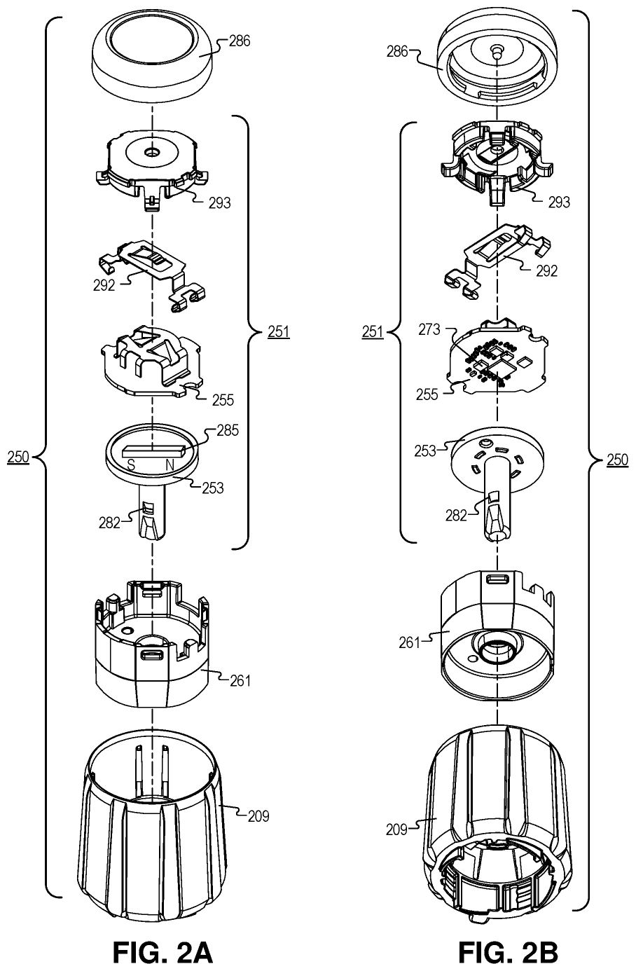

Injection pen 101 is a hand-held device and includes needle 103, body/housing 107 (including a dosage injection mechanism to push in plunger head 115 and extract fluid from drug cartridge 111), and drug delivery control wheel 109 (twist wheel 109 to “click” select the dosage), and pen button 150 (push button 109 with thumb to dispense the selected quantity of the fluid from cartridge 111). It is appreciated that in some embodiments, pen button 150 may include a dosage measurement system (see e.g., FIGS. 2A and 2B) and the drug delivery control wheel 109. As shown, housing 107 is configured to accept cartridge 111: cartridge 111 may be disposed in an insert which screws/snaps onto the bulk of housing 107. However, as one of ordinary skill in the art will appreciate, injection pen 101 can take other configurations and have other components.

As stated, injection pen 101 includes a housing/body 107 shaped to accept a cartridge containing a fluid, and also includes a dosage injection mechanism positioned in the housing 107 to produce a rotational motion and force the fluid out of the cartridge when the drug injection pen 101 dispenses the fluid. A dosage measurement system is also disposed in the pen (e.g., in button 150 or elsewhere in pen body 107) to receive a rotational motion from the dosage injection mechanism. The dosage measurement system may measure a change in magnetic field in a portion of the dosage measurement system, and the dosage measurement system outputs a signal indicative of magnetic field as the drug injection pen 101 dispenses the fluid.

A controller is also disposed in drug injection pen 101, and is coupled to the dosage measurement system. The controller includes logic that when executed by the controller causes the controller to record the electrical signal output from the dosage measurement system when drug injection pen 101 dispenses the fluid. One of ordinary skill in the art will appreciate that the controller may be static (e.g., have logic in hardware), or dynamic (e.g., have programmable memory that can receive updates). In some embodiments, the controller may register the electrical signal output from the dosage measurement system as an injection event of the fluid, and the controller may calculate a quantity of the fluid dispensed based, at least in part, on a number of the injection events of the fluid registered by the controller. It is appreciated that this circuitry, which will be described in greater detail in connection with other figures, may be disposed anywhere in drug injection pen 101 (e.g., in body/housing 107 or pen button 150), and in some instances, logic may be distributed across multiple devices.

Processing device 121 (e.g., a smartphone, tablet, general purpose computer, distributed system, servers connect to the internet, or the like) may be coupled to receive dosage data from injection pen 101 to store/analyze this data. For instance, in the depicted embodiment, processing device 121 is a smartphone, and the smartphone has an application running recording how much insulin has been spent from pen 101. Moreover, the application is plotting how much insulin has been injected by the user over the past week. In this embodiment, a power source is electrically coupled to the controller in injection pen 101, and a transceiver is electrically coupled to the controller to send and receive data to/from processing device 121. Here, data includes information indicative of a quantity of the fluid dispensed. Transceiver may include Bluetooth, RFID, or other wireless communications technologies.

FIGS. 2A-2B illustrate an exploded view of the pen button 250 attachment (e.g., one embodiment of pen button 150 in FIG. 1), in accordance with an embodiment of the disclosure. FIGS. 2A and 2B illustrate the same embodiment of pen button 250, but FIG. 2A illustrates an exploded view looking from the top down, and FIG. 2B illustrates an exploded view looking from the bottom up. It is appreciated that the attachment may have different form factors than the button 250 depicted. Pen button 250 includes drug delivery control wheel 209 (also known as a “dial grip”), housing 261, locking tab 282, pedestal 253, circuit board assembly 255, one or more magnetic sensors 273 (e.g., large brick close to center of circuit board assembly 255, which may include multiple sensors), magnet(s) 285 retaining spring 292, housing clip 293, and spinner 286. As shown, locking tab 282, pedestal 253, circuit board assembly 255, one or more magnetic sensors 273, magnet(s) 285, retaining spring 292, housing clip 293 are disposed in dosage measurement system 251.

In some embodiments, spinner 286 may be made from polybutylene terephthalate (e.g., Celanex 2404MT). Spinner 286 may interact mechanically with (and bear on) housing 261, housing clip 293, and the arm (e.g., center cutout) of retaining spring 292. Housing clip 293 may be made from polycarbonate (e.g., Makrolon 2458). Housing clip 293 may snap fit to housing 261, and housing clip 293 may bear on spinner 286. Pedestal 253 (e.g., a spindle) may also be made from polycarbonate, and snap into a clutch in the pen. Pedestal 253 may also bear on housing 261. Housing 261 may be made from polyoxymethylene (e.g., Hostaform MT8F01). And housing 261 may bear on the clutch (e.g., in the pen body), spinner 286, and the linear slide on the drug delivery control wheel 209. Drug delivery control wheel 209 may also be made from polycarbonate, and it interacts with the linear slide on housing 261.

In operation, the components may move together according to the following steps (discussed from a user-fixed reference frame). A user may dial a dose using drug delivery control wheel 209. The user presses down on spinner 286 with their thumb. Spinner 286 presses housing 261 down. Housing 261 presses the clutch inside the pen body down, and the clutch disengages. Drug delivery control wheel 209 and housing 261 will spin with the circuit board assembly 255 as the drugs are dispensed and pedestal 253/spinner 286 stay rotationally stationary. Thus, drug delivery control wheel 209, housing 261, and circuit board assembly 255 are mechanically coupled to rotate when fluid is dispensed. Tabs on circuit board assembly 255 interact with features on the inside of housing 261 to spin circuit board assembly 255. It is important to note that while dialing a dose, there may be no relative motion between pedestal 253 and circuit board assembly 255, and that while dispensing, circuit board assembly 255 rotates while pedestal 253 is fixed to the user-reference frame.

In some embodiments, pedestal 253 is connected to the clutch (contained in the pen body and included in the dosage injection mechanism)—these parts may not move relative to one another. The clutch is connected to the drive sleeve (also included in the dosage injection mechanism)—which moves axially relative to the clutch with about 1 mm range of motion. The lead screw is threaded into the drive sleeve. If the user has dialed a dose and applies force to button 250, the clutch releases from the numbered sleeve and the lead screw is pushed through a threaded “nut” in the pen body causing the lead screw to advance. When the lead screw advances, it presses on the rubber stopper in the medication vial to dispense fluid.

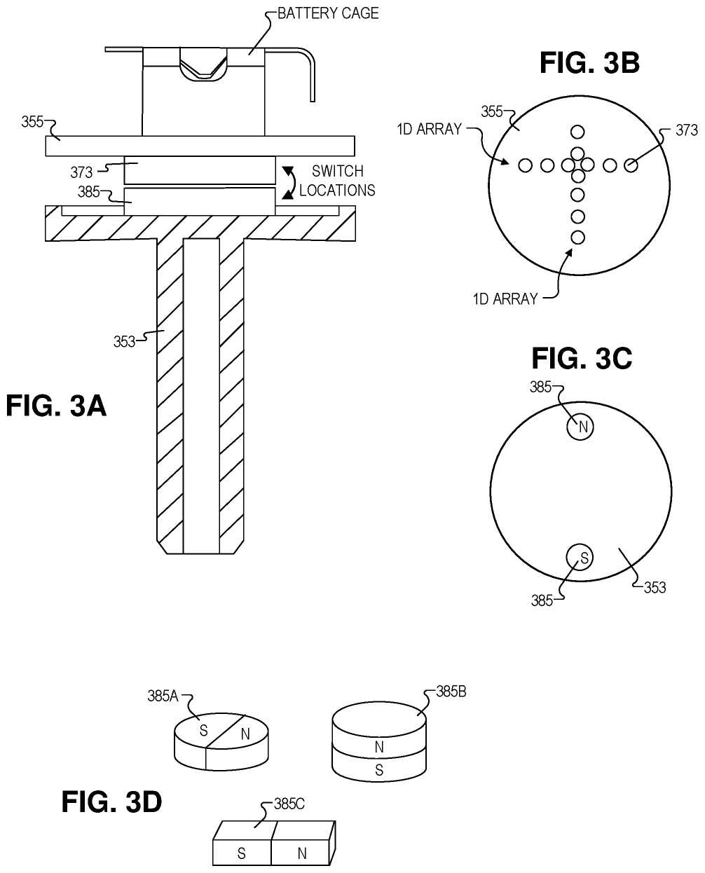

In the depicted embodiment, one or more magnetic sensors 273 (e.g., a TMR sensor, anisotropic magnetoresistance (AMR) sensor, or the like) are positioned along circuit board assembly 255 to measure the magnetic field as the circuit board assembly 255 spins relative to magnets 285 (see e.g., north-south poles resulting in parallel and antiparallel orientation of magnetic fields described above as circuit board assembly 255 rotates). In the depicted embodiment, magnetics sensors 273 are positioned on the same side of circuit board assembly 255 as pedestal 253. In this configuration, magnetic sensors 273 receive a strong magnetic field due to their proximity to magnet 285, thus improving the signal to noise ratio. In some embodiments, magnetic sensors 273 may be placed on components other than circuit board assembly 255 (e.g., magnet 285 could be placed on circuit board assembly 255, and magnetic sensors 273 could be placed on pedestal 253—so that the magnets 285 would spin when fluid is dispensed but magnetic sensors 273 would remain stationary; see e.g., FIG. 3A). One of ordinary skill in the art will appreciate that magnet 285 and magnetic sensors 273 may be placed anywhere in the pen provided there is relative rotational motion between the two so that dosage can be accurately measured.

In some embodiments, the device shown in FIGS. 2A and 2B may be fabricated according to the following steps. The PCB on circuit board assembly 255 may be assembled and programed, and the battery is inserted into the metal cage. Pedestal 253 is inserted into housing 261. Circuit board assembly 255 is inserted into housing 261, and retaining spring 292 is placed on top. Housing clip 293 is snapped into housing 261 above retaining spring 292, and spinner 286 is snapped into housing clip 293. The assembled pen button 250 is then inserted into an assembled pen with a dial grip.

FIGS. 3A-3D illustrate several examples of magnet and magnetic sensor configurations that may be used within the pen button, in accordance with several embodiments of the disclosure. FIG. 3A depicts several (highly simplified) illustrations of pieces of device architecture that correspond to the device architecture depicted in FIGS. 2A and 2B. Shown are pedestal 353, one or more magnets 385, one or more magnetic field sensors 373, circuit board assembly 355, and a battery cage. It is appreciated that a single illustrated block may represent multiple components (e.g., one or more magnets represented by a single block 385).

As illustrated, one or more magnets 385 are disposed on the top circular portion of pedestal 353, and one or more one or more magnetic field sensors 373 are disposed on circuit board assembly 355. However, in some embodiments, one or more magnetic field sensors 373 may be disposed on pedestal 353 and one or more magnets 385 are disposed on circuit board assembly 355 (e.g., illustrated by the double sided arrow and “switch locations”). Circuit board assembly is positioned within the pen button to rotate when the pen dispenses fluid. Thus, magnets 385 and magnetic field sensors 373 rotate relative to one another.

FIG. 3B illustrates one embodiment of a sensor array (including a plurality of magnetic field sensors 373) disposed in two, one-dimensional arrays on circuit board assembly 355. As shown, the two one-dimensional arrays are positioned orthogonal to one another to form a single two-dimensional array. The arrays are offset from each other 90 degrees out of phase (i.e., quadrature encoded). This allows for accurate measurement of the relative position of magnetic sensors 373 to magnets 385. In other embodiments, the 2D array may be formed with a square, rectangular, or circular array of magnetic field sensors 373.

FIG. 3C illustrates one embodiment of a top-down view of a configuration of magnets 385. Here, two axial magnets 285, one with a north pole pointing up and another with a south pole pointing up, are disposed on pedestal 253. Thus, as the magnetic sensors 373 rotate, the magnetic field that the sensors experience will alternate polarity.

FIG. 3D shows several different types of magnets 385 that may be used in the pen button. Depicted are disc magnet 385A, an axial magnet 385B, and a bar magnet 385C. One of ordinary skill in the art will appreciate that the illustrated magnets are just a few different examples of magnets, and that other examples are possible in accordance with the teachings of the present disclosure.

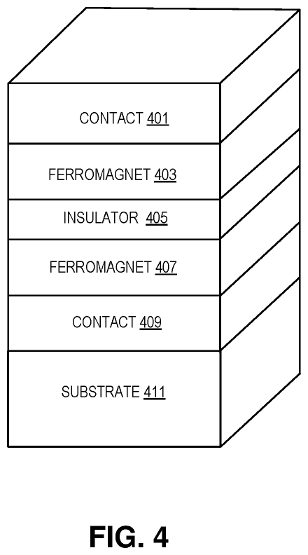

FIG. 4 illustrates an example tunnel magnetoresistance (TMR) device architecture, in accordance with an embodiment of the disclosure. The depicted TMR device architecture includes first contact 401, first ferromagnetic layer 403, insulator 405 (e.g., tunneling layer), second ferromagnetic layer 407, second contact 409, and substrate 411. First contact 401 and second contact 409 may include a conductive metal, and a voltage may be applied across the device. The direction of magnetization of the one or more of the ferromagnetic films 403/407 can be switched in the presence of a magnetic field. If the magnetization of the ferromagnetic films 403/407 is parallel, there is a higher probability that electrons will tunnel through insulator 405 than if the magnetization of the films 403/407 is antiparallel. Accordingly, the resistance changes from very low to very high depending on the direction of the applied magnetic field. This may be used to measure the orientation of the magnet inside the pen button, and subsequently measure dosage with a high degree of accuracy.

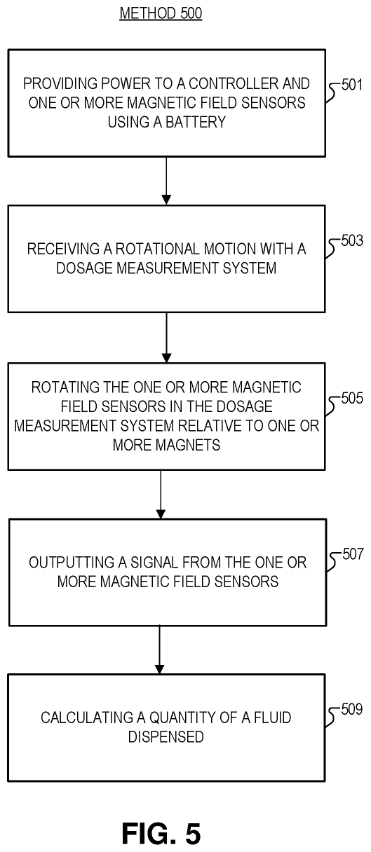

FIG. 5 is a flow chart illustrating a method 500 of dispensing a fluid from a drug cartridge, in accordance with several embodiments of the disclosure. The order in which some or all of process blocks 501-509 appear in method 500 should not be deemed limiting. Rather, one of ordinary skill in the art having the benefit of the present disclosure will understand that some of method 500 may be executed in a variety of orders not illustrated, or even in parallel. Further, blocks may be added to or removed from method 500 in accordance with the teaching of the present disclosure.

Block 501 shows providing power to the controller and the one or more magnetic field sensors using a battery. Power may be provided when the user presses down on the button attached to the proximal end (opposite the distal dispensing end) of the drug injection pen. Pressing on the button may turn on or “wake up” the pen. In some embodiments, the circuit board in the circuit board assembly is disposed between the one or more magnetic field sensors and the battery.

Block 503 illustrates receiving a rotational motion with a dosage measurement system (disposed at least in part in a button attached to a proximal end of the drug injection pen), and the rotational motion is received from a dosage injection mechanism disposed within the drug injection pen. The rotational motion is received when the drug injection pen dispenses a fluid.

Block 505 depicts rotating one or more magnetic field sensors in the dosage measurement system relative to one or more magnets when the drug injection pen dispenses the fluid, and the one or more magnets output a magnetic field. Rotating one or more magnetic field sensors may include rotating a circuit board assembly along with the controller and the one or more magnetic field sensors disposed on the circuit board assembly. In one embodiment, rotating one or more magnetic field sensors may include holding the one or more magnets stationary, relative to the circuit board assembly and the drug injection pen, by positioning the magnets on a stationary pedestal. The magnets may include at least one of a disc magnet, an axial magnet, a bar magnet. The magnetic field may be measured with one or more tunnel magnetoresistance (TMR) devices.

Block 507 shows outputting a signal from the one or more magnetic field sensors indicative of the magnetic field measured by the one or more magnetic field sensors. In some embodiments, outputting the signal includes measuring the magnetic field with the one or more magnetic field sensors when a user presses on the button to determine an initial location of the one or more magnetic field sensors. After measuring the initial rotational location, the device may sample the magnetic field as the one or more magnetic field sensors rotate relative to the one or more magnets when the drug injection pen dispenses the fluid. This may result in the one or more magnetic field sensors outputting a substantially sinusoidal waveform (e.g., a waveform where at least part of the waveform resembles a sine wave—one of ordinary skill in the art will appreciate that the wave may be discontinuous due to the sampling rate, and may include noise, or the like). Since in some embodiments, the magnets are stationary relative to the magnetic field sensors, the sensor's measurements will change as it passes through different regions of the magnetic field.

In some embodiments, outputting the signal includes outputting a signal from a plurality of magnetic field sensors, and the plurality of magnetic field sensors are arranged into a one dimensional array or a two dimensional array.

Block 509 illustrates calculating a quantity of the fluid dispensed using both the initial location of the one or more magnetic field sensors, and the measurement of the magnetic field as the one or more magnetic field sensors rotate relative to the one or more magnets. In one embodiment, the magnetic sensors may be coupled together to form a bridge that outputs two differential analog signals that may be out of phase. Position information about the dosage injection mechanism (e.g., how far the lead screw has turned down into the device—one rotation, two rotations, etc.—and the corresponding dosage expelled) may be determined using a lookup table stored in memory coupled to, or included in, the controller.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. An button attachment for a drug injection pen, comprising:
a housing shaped to attach to a proximal end, opposite a dispensing end, of the drug injection pen; and
a dosage measurement system disposed at least in part within the housing, and coupled to receive a rotational motion, from a dosage injection mechanism disposed within the drug injection pen, when the drug injection pen dispenses a fluid, the dosage measurement system including:
one or more magnets to generate a magnetic field; and
one or more magnetic field sensors to output a signal indicative of an angular position of the one or more magnetic field sensors relative to the one or more magnets, wherein the one or more magnetic field sensors rotate relative to the one or more magnets when the drug injection pen dispenses the fluid.

2. The attachment of claim 1, further comprising a controller included in the dosage measurement system and coupled to the one or more magnetic field sensors, wherein the controller includes logic that when executed by the controller causes the attachment to perform operations, including:
receiving the signal output from the one or more magnetic field sensors, wherein the signal includes a substantially sinusoidal component; and
calculating a quantity of the fluid dispensed based at least in part on the signal.

3. The attachment of claim 2, further comprising a circuit board assembly including the controller, and the one or more magnetic field sensors, wherein the circuit board is coupled to receive the rotational motion and rotate the circuit board assembly relative to the one or more magnets.

4. The attachment of claim 3, further comprising a pedestal disposed at least in part in the housing, wherein the one or more magnets are disposed on the pedestal, and wherein the pedestal is positioned to remain stationary relative to the circuit board assembly when the drug injection pen disposes the fluid.

5. The attachment of claim 3, further comprising a battery coupled to provide power to the controller and the one or more magnetic field sensors, wherein a circuit board in the circuit board assembly is disposed between the one or more magnetic field sensors and the battery.

6. The attachment of claim 5, wherein the battery is disposed in a cage attached to the circuit board assembly.

7. The attachment of claim 1, wherein the one or more magnetic field sensors comprises a plurality of magnetic field sensors, and the plurality of magnetic field sensors are arranged into a one dimensional array or a two dimensional array.

8. The attachment of claim 9, wherein the plurality of magnetic field sensors are arranged into two one-dimensional arrays positioned orthogonal to one another.

9. The attachment of claim 1, wherein the one or more magnetic field sensors include one or more tunnel magnetoresistance (TMR) devices.

10. The attachment of claim 1, wherein the one or more magnets include at least one of a diametric magnet, an axial magnet, or a bar magnet.

11. The attachment of claim 1, further comprising a spinner disposed on the proximal end of the button attachment, wherein the spinner is positioned to remain stationary under a thumb of the user when the drug injection pen dispenses the fluid.

12. A method of measuring a fluid dispensed from a drug injection pen, comprising:
receiving a rotational motion with a dosage measurement system disposed at least in part in a button attached to a proximal end of the drug injection pen, wherein the rotational motion is received from a dosage injection mechanism disposed within the drug injection pen when the drug injection pen dispenses a fluid;
rotating one or more magnetic field sensors in the dosage measurement system relative to one or more magnets when the drug injection pen dispenses the fluid, wherein the one or more magnets generate a magnetic field; and
outputting a signal from the one or more magnetic field sensors indicative of the magnetic field measured by the one or more magnetic field sensors.

13. The method of claim 12, wherein outputting the signal, includes:
measuring the magnetic field with the one or more magnetic field sensors when a user presses on the button to determine an initial location of the one or more magnetic field sensors; and
measuring the magnetic field as the one or more magnetic field sensors rotate relative to the one or more magnets when the drug injection pen dispenses the fluid.

14. The method of claim 13, wherein rotating one or more magnetic field sensors includes rotating a circuit board assembly including a controller and the one or more magnetic field sensors.

15. The method of claim 14, wherein rotating one or more magnetic field sensors includes holding the one or more magnets stationary, relative to the circuit board assembly and the drug injection pen, on a pedestal.

16. The method of claim 14, further comprising providing power to the controller and the one or more magnetic field sensors using a battery, wherein a circuit board in the circuit board assembly is disposed between the one or more magnetic field sensors and the battery.

17. The method of claim 13, further comprising calculating a quantity of the fluid dispensed using both the initial location of the one or more magnetic field sensors, and the measurement of the magnetic field as the one or more magnetic field sensors rotate relative to the one or more magnets.

18. The method of claim 13, wherein outputting the signal includes outputting a substantially sinusoidal waveform as the one or more magnetic field sensors rotate relative to the one or more magnets.

19. The method of claim 12, wherein outputting the signal includes outputting a signal from a plurality of magnetic field sensors including the one or more magnetic sensors, and the plurality of magnetic field sensors are arranged into a one dimensional array or a two dimensional array.

20. The method of claim 12, further comprising measuring the magnetic field with one or more tunnel magnetoresistance (TMR) devices included in the one or more magnetic field sensors.

21. The method of claim 12, wherein outputting the magnetic field includes using at least one of a disc magnet, an axial magnet, or a bar magnet.
